(19) United States
(12) Patent Application Publication
(10) Pub. No.: US 2006/0040273 A1
(43) Pub. Date: Feb. 23, 2006

(54) Method and apparatus for magnetic sensing and control of reagents

(76) Inventors: Alison Chaiken, Fremont, CA (US); Manish Sharma, Sunnyvale, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/919,968

(22) Filed: Aug. 17, 2004

(51) Int. Cl. C12Q 1/68; C12M 1/34

(52) U.S. Cl. 435/6; 435/287.2

(57) ABSTRACT

An apparatus for characterizing reactions including a spinnable medium with one or more internal chambers capable of containing one or more reagents, a composite reagent that includes a magnetic component, a rotating mechanism capable of turning the spinnable medium, and a reading mechanism capable of measuring the magnetic component at one or more regions of the spinnable medium.

### BACKGROUND

The present invention relates to microfluidic materials. Research using microfluidic materials is widespread in a variety of fields, including medicine, chemistry, biology, and genetics. Microfluidic based genomic and proteomic assays using functionalized arrays and fluorescent proteins have become standard tools of modem biotechnology. Moreover, microfluidics are increasingly used by medical laboratories, physicians, and even with individual patients in conjunction with various treatment and diagnosis.

Unfortunately, reading the microfluidic assays currently requires expensive scanners. For example, a confocal scanner is expensive as it images microscopic samples one “point” at a time by spatially confining the detected light. While these expensive scanner devices may be affordable in a hospital or laboratory setting, the excessive cost discourages wide adoption among physicians and their patients. Clearly, a less-expensive alternative would facilitate inexpensive research, rapid point-of-care testing, and even home health evaluation.

Recently, several research groups have proposed an inexpensive micro-analytical system based on a compact disk (CD) player of the type found in personal computers. The basic idea of this “integrated bio compact disk” (IBCD) technology is to use the rotary motion of a disk drive for centrifugal separation and the CD player's laser optics to read the results. The experimenter incorporates the microfluidic test materials into a disk having the dimensions of a compact disk and then runs the experiment.

Conventional IBCD reactions incorporate “recognition molecules” created as a result of an experiment and then sensed using optical sensors. The IBCD system optically reads the results by observing whether the recognition molecules are bound after the reactions occur and where the bonds are located. IBCD systems currently use the optics in several different ways to detect the recognition molecules. For instance, the laser and photosensor of the CD player detects a change in the light transmission through an optical waveguide placed parallel to the surface of the disk. Alternatively, the photosensor detects changes in the transmission and reflection of light in a test chamber.

However, the conventional measurement techniques used in IBCD systems contain many disadvantages. For example, turbidity of the analyte can disrupt the optical read back as a result of particles or other material suspended in the solution. This often leads to false indications of reaction when bindings are not taking place, and vice versa. Moreover, the mathematical techniques used to predict the flows of fluids within the device requires the disk to operate with a high degree of precision and predictability. Without these qualities, designing complex reaction sequences using conventional IBCD technology is quite difficult.

Another problem is the tendency of volatile reagents in an IBCD disk to evaporate during storage. Consequently, it is possible that a vapor may disperse through the whole system even if the liquid portion of the reagent is restricted from flowing through the various microfluidic channels. Unfortunately, the result could cause a reagent could lose its solvent or change composition. In addition, air permeating the system could react destructively with reagents.

Although IBCD represented a cost savings over prior laboratory techniques, the aforementioned disadvantages limit its applicability. New techniques for sharing IBCD's advantages while simultaneously avoiding its shortcomings would make the benefits of microfluidic experimentation more cost effective and available to a wider group of users.

### BRIEF DESCRIPTION OF THE DRAWINGS

The present invention is illustrated by way of example and not limitation in the figures of the accompanying drawings in which:

FIG. 1 is a schematic illustrating the incorporation of a laboratory experiment into a disk for use in one embodiment of the present invention;

FIG. 2A is a schematic showing the introduction of a target reagent into a reaction chamber containing a compound reagent for use in a sandwich assay in accordance with one embodiment of the present invention;

FIG. 2B is a schematic of a chemical reaction taking place during a sandwich assay performed by one embodiment of the present invention;

FIG. 3 is a flowchart of operations for performing a sandwich assay in accordance with one embodiment of the present invention;

FIG. 4A is a schematic of a magnetically actuated valve directing fluid down a first output channel in accordance with one embodiment of the present invention;

FIG. 4B is a schematic of a magnetically actuated valve directing fluid down a second output channel in accordance with one embodiment of the present invention;

FIG. 4C is a schematic of an embodiment of the present invention capable of controlling valves connecting chambers in a spinnable medium;

FIG. 5 is a flowchart of operations controlling the flow of fluids in one embodiment of the present invention; and

FIG. 6 is a block diagram of a system used in controlling the apparatus or methods in accordance with one embodiment of the present invention.

### SUMMARY OF THE INVENTION

One aspect of the present invention describes an apparatus for characterizing reactions. The apparatus includes a spinnable medium with one or more internal chambers capable of containing one or more reagents, a composite reagent that includes a magnetic component, a rotating mechanism capable of turning the spinnable medium, and a reading mechanism capable of measuring the magnetic component at one or more regions of the spinnable medium.

Another aspect of the present invention describes a method of regulating the flow of fluids in a spinnable medium. The method includes inserting a magnetic material into valve areas that separate channels capable of carrying fluids in a spinnable medium, selectively introducing a magnetic field gradient in the vicinity of the valve areas to displace the magnetic material associated, opening a connection between one or more of the channels responsive to the displacement of the magnetic material, and rotating the spinnable medium so that fluids flow through the valve areas under the influence of centrifugal force.

### DETAILED DESCRIPTION

Aspects of the present invention provide at least one or more of the following advantages. Embodiments of the present invention provide an integrated reaction analysis and detection system based on existing mass-produced magnetic storage technologies. Advancements in magnetic storage technologies enable embodiments of the present invention to measure lower concentrations of marker molecules than current techniques. Further, resulting measurements are less susceptible to disturbance by background effects such as solution turbidity as particulate matter in the solution does not interfere with the magnetic field emanating from the magnetically marked molecules. Through the use of inexpensive and readily available mass-produced consumer technologies, microfluidic based analysis using embodiments of the present invention are now accessible to point-of-care providers and even home users.

Moreover, embodiments of the present invention enable precise control of valves and pumps for microfluidic experiments. Increased control over these valves and pumps leads to greater flexibility in designing experiments. For example, precise control over microfluidics enables manufacturers to prepare and pre-load chambers with various reagents for various microfluidic experiments and then distribute for later use.

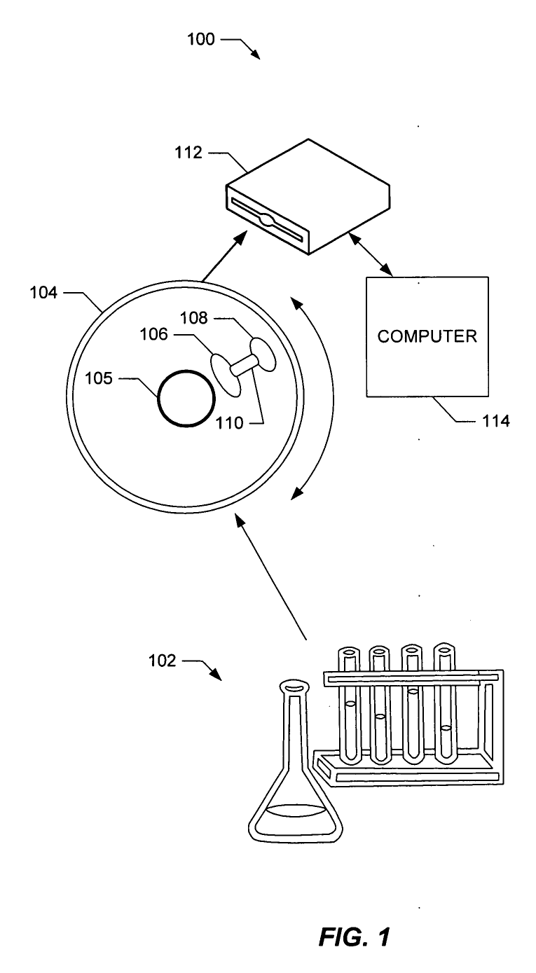

FIG. 1 is a schematic illustrating the incorporation of a laboratory experiment into a disk for use in one embodiment of the present invention. System 100 includes a reaction 102, a spinnable medium 104, a reaction chamber 106, an output chamber 108, a channel 110, a rotating mechanism 112, and a computer 114.

Reaction 102 represents the occurrence of a microfluidic reaction. Today, experimental and clinical microfluidic measurements are already in widespread use. The reaction can range from DNA sequencing, enzyme activity assays, and proteomics analysis to diagnostic microarrays and immuno sensing. In this particualr example, reaction 102 represents a chemical “sandwich assay,” as described in more detail below. Alternate embodiments of the present invention can be adapted to work with many other experimental configurations.

Spinnable medium 104 contains reservoirs of fluid reagents positioned so that varying the rotation speed around a center axis 105 allows sequencing of the flow of the fluids. Materials farther from center axis 105 experiences the strongest centrifugal forces and flow first provided all other parameters (e.g., viscosity and channel width) are equal. Various microfluidic mathematical models are constructed to predict flows through various channels of the device. In one embodiment of the present invention described in further detail below, magnetically actuated valves regulate these flows.

In the present embodiment, spinnable medium 104 has substantially the shape of a commercially available removable magnetic information storage disk. However, unlike commercially available disks, the spinnable medium contains a reaction chamber 106, an output chamber 108, and a connecting channel 110 in accordance with one embodiment of the present invention.

Spinnable medium 104 can be constructed in a variety of ways. For example, it can be constructed from a non-magnetic plastic laminate disk consisting of several layers created by injection molding, by milling or by soft lithography. Alternatively, spinnable medium 104 is constructed from multiple individually formed plastic layers. In this latter embodiment, spinnable medium 104 has a smoother surface and characteristic lubrication that are compatible with the reading mechanism, described below.

Optionally, spinnable medium 104 also includes areas coated with a ferromagnetic material capable of storing information. This material operates much like a standard magnetic storage device. For example, the magnetic coating may be initialized to include information about the operation of the experiment while later it can be used to store the results of the experiment.

In the present embodiment, an experimenter loads reaction chamber 106 with a compound reagent (not shown) further including two additional components: a tethered component and a magnetic component. The first component is tethered to the inner surface of the reaction chamber 106 through a chemical, biochemical and/or mechanical bond (i.e., surface tension). In turn, the second magnetic component bonds weakly with the tethered component through another chemical, biochemical and/or mechanical bond with the tethered component. In one embodiment of the present invention, the tethered component includes a DNA strand that is the target of an experimental drug. In another embodiment, the tethered component includes an expressed sequence tag and the magnetic component includes a cDNA made from the mRNA of a patient's cells. In this latter case the embodiments of the present invention can be used to study gene expression. It is contemplated that embodiments of the present invention can be applied to many other component reagent combinations.

The experimenter then adds a target reagent (not shown) to reaction chamber 106. Reaction chamber 106 can be constructed with a latex material or any other semi-permeable barrier that the target reagent can be injected through. In another possible embodiment, a small hole on the interior wall lining central axis 105 serves for introducing the target reagent into reaction chamber 106.

The target reagent displaces the magnetic component when the target reagent's bond to the tethered component is stronger than the bond of the magnetic component. Alternatively, only a portion of the magnetic component is displaced in correlation to the relative strength of target reagent to the magnetic component's bond. In either case, the displaced magnetic component is then free to move in reaction chamber 106 potentially going through connecting channel 110 and onto output chamber 108.

The experimenter then inserts spinnable medium 104 into rotating mechanism 112. Rotating mechanism 112 contains a reading mechanism capable of both generating and sensing magnetic fields at arbitrary regions of the spinnable medium. In the embodiment shown, the rotating mechanism is based upon a commercially available removable magnetic information storage device. For example, commercially available magnetic information devices include removable hard-drives, floppy drives and other storage mediums. These devices are remarkably inexpensive yet sophisticated instruments for manipulating, reading from, and writing to spinnable medium 104.

Computer 114 controls operation of rotating mechanism 112. Although rotating mechanism 112 shown may appear unaltered, underlying drivers in computer 114 contain one or more specialized routines that facilitate controlling and reading experimental results. For example, the commercially available magnetic information devices also may have slightly modified firmware in order to permit more complex sequences of head motions and rotations than off-the-shelf units. Computer 114 is also likely to contain other software related to performing the experiment. For example, computer 114 may also contain routines for analysis and tracking of biochemical reagents and processing of the particular experimental results.

Under the control of computer 114, rotating mechanism 112 turns spinnable medium 104. Centrifugal force causes the free reagents to flow from reaction chamber 106 to output chamber 108. If reaction 102 has freed the magnetic component from its bond to the tethered component, the magnetic component will exit reaction chamber 106 through connecting channel 110 and into output chamber 108.

The reading mechanism then determines the relative distribution of the magnetic component in the reaction chamber 106 and output chamber 108. Comparing the measurements made before reaction 102 with the measurements made after reaction 102 provides important information. In many cases, the experimenter's measurements are used to directly determine various experimental results of the reaction.

It should be appreciated that many other arrangements of microfluidic chambers, channels, valves and reagents are also possible. In one or more embodiments of the present invention, chambers on spinable medium 104 are isolated from one another via one or more ferrofluidic valves. Embodiments of the present invention toggle the ferrofluidic valves at appropriate times during the analysis, as described in more detail below. In addition to those previously described, spinnable medium 104 may contain many other components. For instance, the spinnable medium may contain waste disposal compartments, or lyophilized reagents that are mixed with a solvent, usually water, as needed.

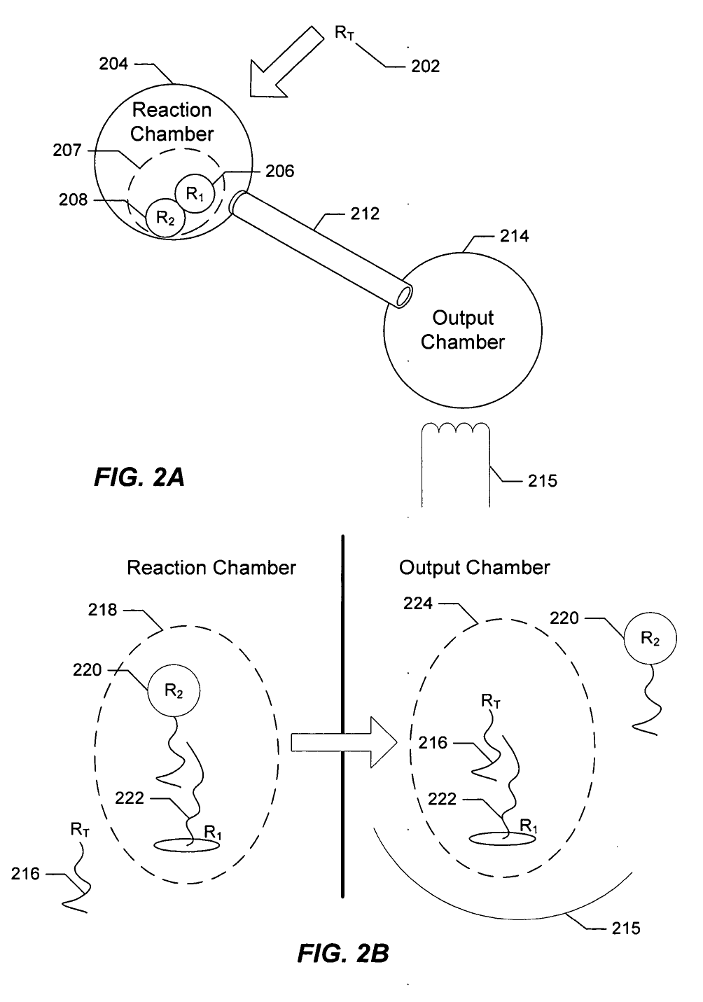

FIG. 2A is a schematic showing the introduction of a target reagent (illustrated as R_{T}) 202 into a reaction chamber 204 containing a compound reagent 207 for use in a sandwich assay in accordance with one embodiment of the present invention. The embodiment as illustrated includes target reagent 202, reaction chamber 204, compound reagent 207 that includes a magnetic component (illustrated as R₂) 206 and a tethered component (illustrated as R₁) 208, a channel 212, and an output chamber 214 all operating under the influence of a magnetic read-write head 215.

Target reagent 202 can used in a variety of experimental contexts and with a variety of materials. For instance, these materials can be used in conduction with performing experiments in genetic engineering or drug design. In the case of gene expression studies, target reagent 202 in one embodiment of the present invention is a cDNA made from patient mRNA that binds to an expressed sequence tag that is part of tethered component 208. In the general case of drug design, target reagent 202 in one embodiment of the present invention is an experimental drug that binds to a protein that is part of tethered component 208.

In the embodiment shown, the experimenter introduces target reagent 202 into reaction chamber 204. Each of the various chambers are of a size and shape conducive to performing the experiment at hand. Consequently, while reaction chamber 204 and output chamber 214 are schematically represented here as spheres, these chambers may in fact be any shape contained in the dimensions of the spinnable medium.

Further, read-write head 215 is represented schematically as a coil even though the actual shape and size may be different. For example, read-write head 215 can be constructed as a single device combining both a magnetic read head (typically a magnetoresistive sensor) and a magnetic write head (typically an inductive write head) so they move together over the top of a disk. Even though read-write head 215 may appear as a single device, the magnetic read head and the magnetic write head typically have separate connection terminals and circuitry but are manufactured as a single device. It is also contemplated that read-write 215 is a very small device on the order of 1000 or more times smaller when compared to either reaction chamber 204 or output chamber 214. Alternatively, read-write head 215 could be replaced or complemented with a permanent magnet that operates like the write head portion of read-write head 215 actuating one more ferrofluid valves designed in accordance with embodiments of the present invention as described later herein.

Tethered component (R₁) 208 is bonded to the interior of the reaction chamber as previously described. Magnetic component 206 is in turn weakly biochemically bound to tethered component (208). In addition to the organic portion of the molecule that binds to the tethered component, magnetic component 206 includes ferromagnetic or paramagnetic beads in accordance with one implementation of the present invention. Such magnetic beads are often used as in marker (i.e., recognition) molecules. They are available in various microscopic sizes and can be functionalized in many ways. For example, magnetic beads may be functionalized by including antigens, expressed sequence tags, cDNAs, proteins, secondary antigen particles, nucleic acids, and amine-terminated particles. By varying the magnetic beads' size, weight, and magnetic moment, an experimenter can alter their behavior under the influence of a magnetic force, gravitational force, or centrifugal force.

If target reagent 202 bonds more strongly to tethered component 208 than magnetic component 206 does then magnetic component 206 will be displaced. By rotating the spinnable medium, an embodiment of the present invention causes magnetic component 206 to flow through channel 212 into output chamber 214.

Magnetic read head 215 then measures the amount of magnetic material in output chamber 214. This in turn characterizes the reaction between the target reagent 202 and tethered component 208.

FIG. 2B is a schematic of a chemical reaction taking place during a sandwich assay performed by one embodiment of the present invention. The schematic includes a target reagent 216, a pre-reaction composite reagent 218 including a magnetic component 220 and a tethered component 222, a post-reaction composite 224, and a magnetic read head 215.

Magnetic component 220 is again weakly bound to tethered component 222 in a reaction chamber. The experimenter introduces target reagent 216 (R_{T}) into the reaction chamber, where it either succeeds or fails to displace magnetic component 220 (R₂) from its bond with tethered component 222 (R₁). In this case, target reagent 216 succeeds in displacing magnetic component 220. Target reagent 216 and tethered component 222 form a post-reaction composite 224.

Embodiments of the present invention then move magnetic component 220 from the reaction chamber an output chamber using centrifugal force. Magnetic read head 215 reads the result of the reaction by sensing the amount of magnetic material remaining in the reaction chamber or, alternatively, now in the output chamber.

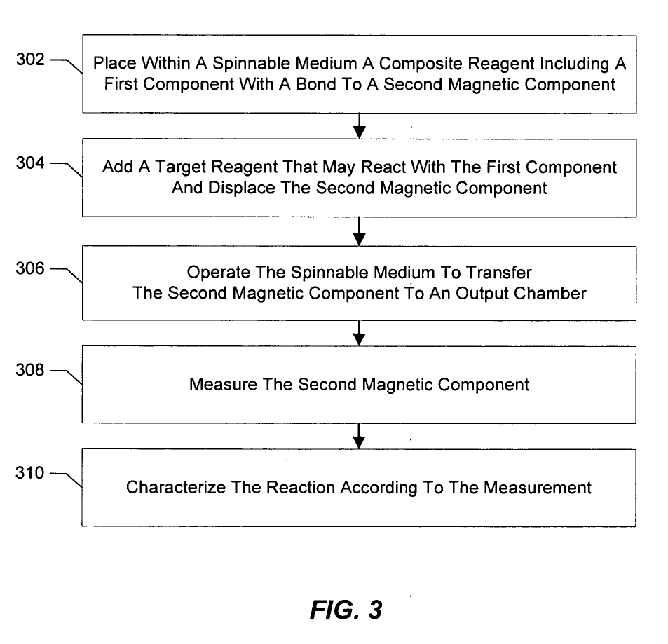

FIG. 3 is a flowchart of operations for performing a sandwich assay in accordance with one embodiment of the present invention. The first operation is to place a composite reagent (302) within a spinnable medium. As described above, the composite reagent includes a first component with a bond to a second magnetic component. The first component is tethered to the inside of the reaction chamber, for instance, by a chemical force.

In one embodiment of the present invention, the magnetic component is a marker detectable through inductance or variable resistance by a nearby read head. In alternative embodiments, different markers sometimes signal a reaction. However, magnetic markers have numerous advantages compared with other types of markers in that the magnetic fields used by the marker tend to be relatively undisturbed by the properties of most chemical solutions.

In one embodiment, the composite reagent is placed in the spinnable medium in two steps: First, the experimenter adds to the reaction chamber a first component that tethers itself to the interior of the reaction chamber. Second, the experimenter adds to the reaction chamber a magnetic component that then bonds to the first tethered component.

The next operation is to add a target reagent that may react with the first component and displace the second magnetic component (304). As previously described, the target reagent displaces the magnetic component when the bond between the target reagent and the first component is relatively stronger than the bond between the magnetic component and the first component.

The spinnable medium next operates to effectuate the transfer of the second magnetic component to an output chamber (306). As described before, various forces and principles can contribute to the flow of fluids in the apparatus. Centrifugal force, rotational acceleration, gravity, and capillary force can each play a role in this process. Therefore, various operations of the spinnable medium can also control the flow of the fluids. For example, the medium can rotate, reciprocate, accelerate, or decelerate. Alternatively, these centrifugal forces could be combined with the magnetic field force from the write head to further help control the flow of materials through the different chambers. For example, the magnetic field from the write head could be used to “sweep out of a chamber” or “guide through channels” any magnetic material that becomes untethered (either initially or after a reaction). These principles, along with the valves described below, allow great flexibility in the design of diagnostic instruments.

After introduction of a target reagent, one embodiment of the present invention then rotates the spinnable medium at a speed that prepares the sample for analysis. This preparation may include many forms of processing, including for example mixing, or centrifugal separation of proteins. In the present example, however, the spinnable medium only uses centrifugal force to cause any free-flowing materials to move to an output chamber.

The next operation measures the second magnetic component (308). Fortunately, the second magnetic component can be measured in a variety of different ways. For example, the magnetic read head of an information storage device can sweep across the surface of the spinnable medium near the output chamber to evaluate the magnetic component there. Conversely, the read head can be used to measure the amount of magnetic component remaining in the reaction chamber. In more complex experiments the apparatus measures the distribution of the magnetic component throughout the spinnable medium rather than one chamber or the other.

The next operation characterizes the reaction according to the measurement of the magnetic component (310). In the case of the simple sandwich assay described above, this characterization determines whether the target reagent bonded to the first component of the compound reagent. For example, the experiment may be to determine whether an experimental drug successfully targets a particular gene or protein.

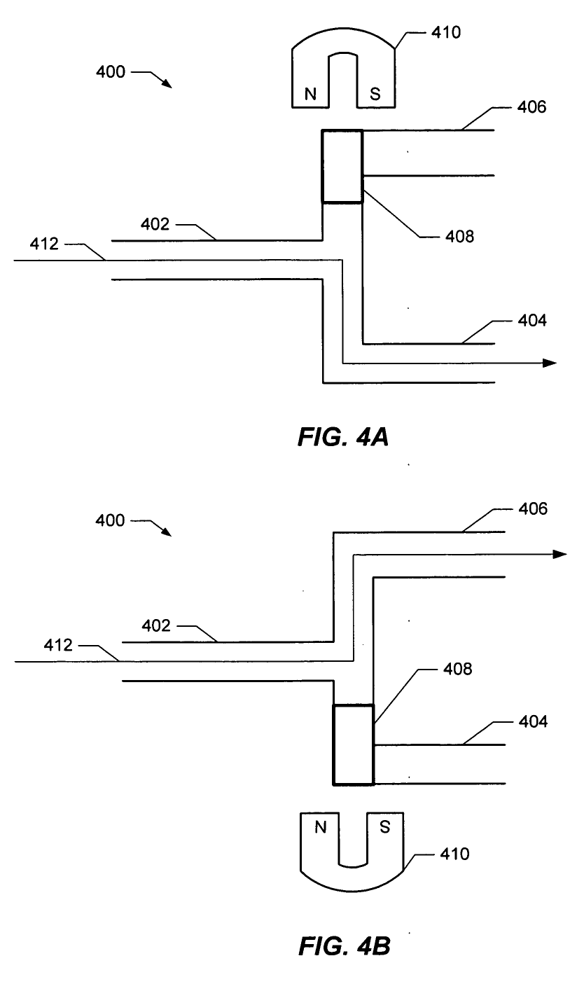

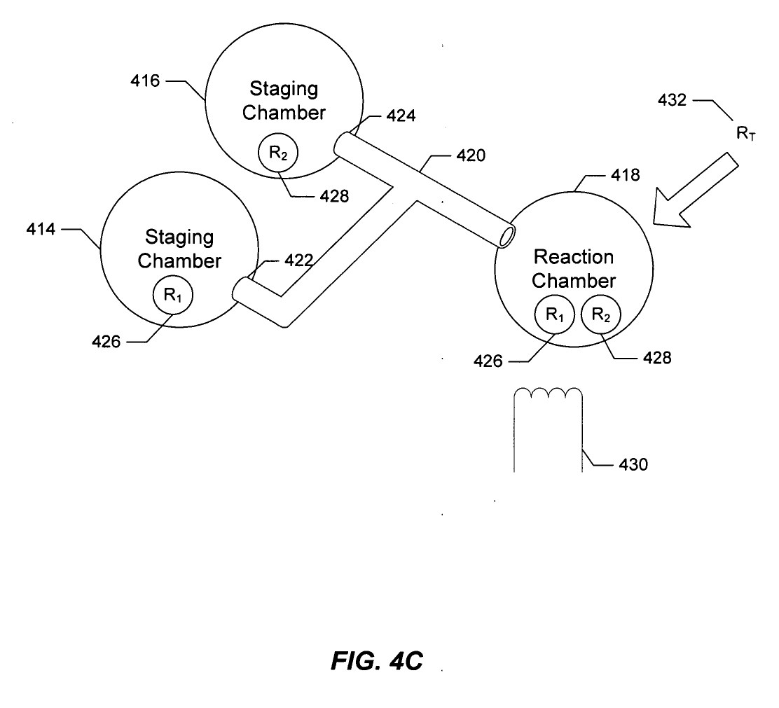

FIG. 4A is a schematic of a magnetically actuated valve 400 directing fluid down a first output channel in accordance with one embodiment of the present invention. Magnetically actuated valve 400 includes an input channel 402, a first output channel 404, a second output channel 406, a moveable magnetic plug 408, a magnet 410, and a fluid 412.

The channels in magnetically actuated valve 400 can be manufactured by many different techniques. In one embodiment, soft lithography etches the channels into one layer of a multi-layer plastic disk. The channels can be oriented so that rotation of the disk causes the fluid to flow in the desired direction under the influence of centrifugal force.

In the illustrated embodiment in FIG. 4A, input channel 402 selectively passes microfluids through either first output channel 404 or second output channel 406 into one of two reaction chambers for two different analytical operations. However, it is contemplated that the channels in magnetically actuated valve 400 can be configured for use in a limitless number of other possible operations. For example, an alternate embodiment of the present invention uses magnetically actuated valve 400 along with one or more chambers preloaded with reagents prior to shipment to a laboratory. Magnetically actuated valve 400 can be used in various combinations to prevent leakage and mixing of the reagents in the spinnable medium prior to use. Furthermore, the ferrofluid acts as a vapor barrier that prevents evaporation or oxidation of the reagents. Without such a seal within the spinnable medium, reagents might disperse throughout the system in the gas phase, causing undesirable reactions and shelf-life problems. In contrast, a ferrofluid seal designed in accordance with embodiments of the present invention can be opened and closed multiple times.

Moveable magnetic plug 408 can likewise be implemented using a variety of different structures. For example, moveable magnetic plug 408 can be implemented as a drop of viscous ferrofluid containing iron, cobalt, nickel or their oxides that operates to plug the channel. Alternatively, the ferrofluid is not used to plug the channel directly but instead is used indirectly to hold a pellet in place that plugs the channel.

In accordance with one embodiment of the present invention and as previously described, magnet 410 is the electromagnetic read/write head of a commercially available information storage device. Each valve can be operated independently and sequentially by magnet 410 located on either or both sides of a platter. Accordingly, one embodiment may have a total of two heads, one head on each side of the medium that operate independently from each other. Alternate embodiments may also be created that have more than one head on each side of the medium. The additional number of heads on each side of the platter may be more costly yet may have additional benefits when used in conjunction with each other. Yet another embodiment could implement magnet 410 using permanent magnets instead of or in combination with read/write heads as previously described.

In either of these or other embodiments, opening and shutting valves facilitates flow sequencing, cascade micro-mixing, and capillary metering by positioning one or more movable magnetic plugs 408 to the control the fluids. An alternate embodiment uses one or more magnet 410 together to emit a single and relatively large diffuse magnetic field that controls all of the valves simultaneously. The single magnetic field directed at one or more magnetically actuated valve 400 on the spinnable medium operates to open one or more of the valves at approximately the same time interval rather than independently as previously described. For example, this operation could be used to ‘break the seal’ on an experiment preloaded into the spinnable medium by a laboratory or manufacturer.

In the embodiment shown in FIG. 4A, magnet 410 has attracted moveable magnetic plug 408 to block second output channel 406 and open first output channel 404. This in turn allows fluid 412 to flow through first output channel 404.

FIG. 4B is a schematic of a magnetically actuated valve 400 directing fluid 412 down a second output channel 406 in accordance with one embodiment of the present invention. Magnetically actuated valve 400 includes an input channel 402, a first output channel 404, a second output channel 406, a moveable magnetic plug 408, a magnet 410, and a fluid 412.

In the embodiment shown in FIG. 4B, magnet 410 has attracted moveable magnetic plug 408 to block first output channel 404 and open second output channel 406. This in turn frees fluid 412 to flow through second output channel 406.

FIG. 4C is a schematic of an embodiment of the present invention capable of controlling valves connecting chambers in a spinnable medium. The schematic includes a first staging chamber 414, a second staging chamber 416, a reaction chamber 418, a channel 420, a first magnetic valve 422, a second magnetic valve 424, a first reagent 426, a second reagent 428, a magnet 430, and a target reagent 432.

In the present embodiment, first staging chamber 414 contains a first reagent 426 (labeled R₁); second staging chamber 416 contains a second reagent 428 (labeled R₂). Target reagent 432 can be introduced directly into reaction chamber 418 or by way of a different set of chambers, valves and channels (not shown), or by direct injection, as previously described.

First reagent 426 and second reagent 428 are held in their respective chambers by a first magnetic valve 422 and a second magnetic valve 424. These valves contain magnetically actuated valves as previously described (not shown). Magnet 430 controls the valves by applying magnetic forces to the magnetically actuated valves also as previously described. In one embodiment of the present invention, magnet 430 is at a fixed azimuth near the spinnable medium and can move radially in order to operate the magnetically actuated valves as needed. Positioning operations or software designed in accordance with implementations of the present invention position a write head to address each valve individually, as previously described.

In the example illustration, staging chamber 416 and staging chamber 414 can be selectively connected to reaction chamber 418 by way of channel 420. A traversing channel connecting staging chamber 414 to channel 420 may be situated at a slight angle to help precipitate the flow of a fluid under the applied centrifugal force. In operation, first magnetic valve 422 and second magnetic valve 424 are partially or completely opened through application of a magnetic field by magnet 430. The experiment occurs when first reagent 426 and second reagent 428 flow through channel 420 to the reaction chamber and combine with target reagent 432 previously or simultaneously introduced into reaction chamber 418. In an alternative embodiment, R₁ and R₂both initially flow in and becomes chemically tethered to each other in reaction chamber 418. In addition to the chemical bond to R₁, R₂ also is tethered to a magnetic bead. Once the reactants R₁ and R₂ settle and create the sandwich assay, Rₜis then introduced whereupon it potentially may displace R₂.

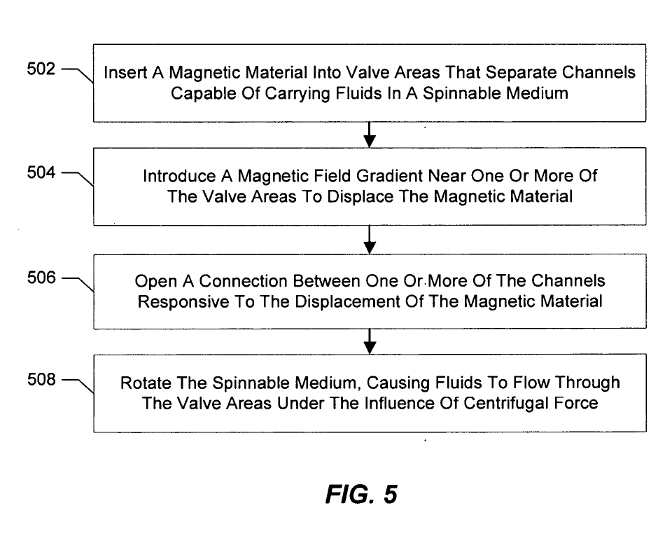

FIG. 5 is a flowchart of operations for controlling the flow of fluids in one embodiment of the present invention. The first operation is to insert a magnetic material into valve areas that separate channels capable of carrying fluids in a spinnable medium (502). The magnetic material can be one of many types. For example, it may be a viscous ferrofluid containing ferrous particles. These particles in turn can be various types. For example, they may be Iron, nickel, Cobalt, their alloys, Ferrous Oxide, or Fe₃O₄ or other magnetic oxides.

The next operation is to introduce a magnetic field gradient near one or more of the valve areas to displace the magnetic material (504). The term “valve” as used here can encompass many types of devices capable of controlling the flow of fluids.

In some embodiments, a connection opens between one or more of the channels responsive to the displacement of the magnetic material (506). In one possible embodiment, the valves may include a magnetic material that directly plugs a valve area between an input channel and one or more output channels. In another, the valve areas operate under indirect control of the ferrofluidic material moving and creating a vacuum that moves solid plugs in the valve areas.

The embodiment then rotates the spinnable medium, causing fluids to flow through the valve areas under the influence of centrifugal force (508). Other embodiments are possible which make use of other principles and forces. For example, capillary motion forces may cause the fluids to flow through the valve areas. A magnetically actuated plug made of biocompatible liquids or solids may be used to push reagents through a channel.

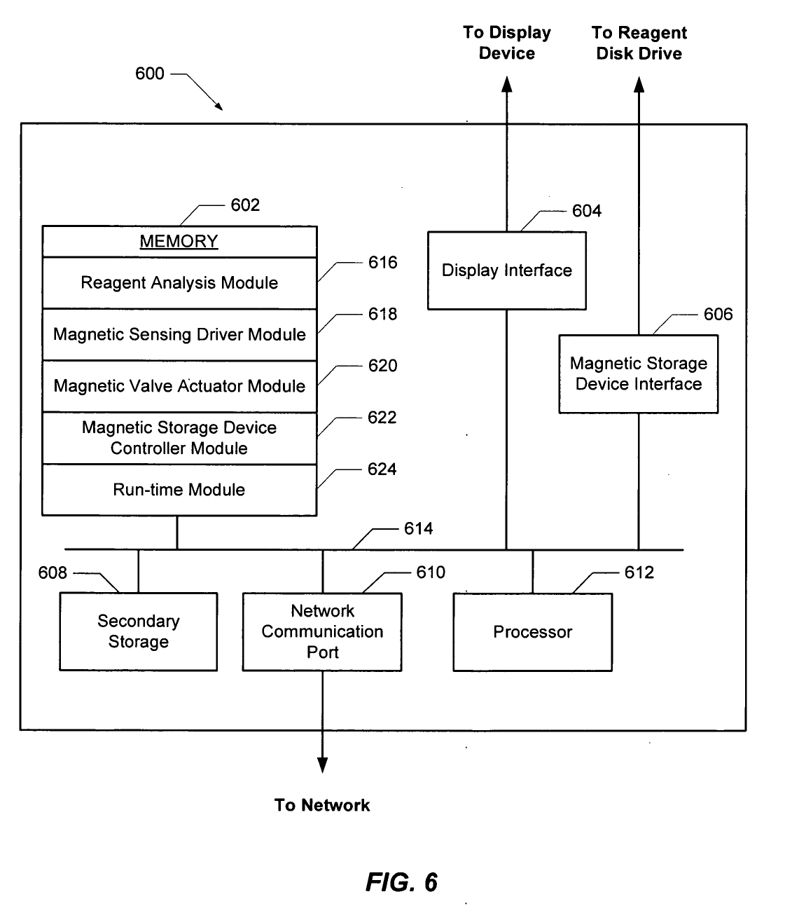

FIG. 6 is a block diagram of a system used in controlling the apparatus or methods in accordance with one embodiment of the present invention. System 600 includes a memory 602 to hold executing programs (typically an ordinary disk drive, random access memory (RAM) or read-only memory (ROM) such as Flash), a display interface 604, a magnetic storage device interface 606, a secondary storage 608, a network communication port 610, and a processor 612, operatively coupled together over an interconnect 614.

Display interface 604 allows presentation of information related to the experiment on an external monitor. Magnetic storage device interface 606 contains circuitry to control of the rotating, reading, and writing mechanisms operating on a spinnable medium. In one embodiment of the present invention, these mechanisms are contained in a commercially available disk-drive or other type of magnetic storage device. Secondary storage 608 can contain experimental results and programs for long-term storage. Network communication port 610 transmits and receives results and data over a network. Processor 612 executes the routines and modules contained in memory 602.

In the illustrated embodiment of the present invention, memory 602 includes a reagent analysis module 616, a magnetic sensing driver module 618, a magnetic valve actuator module 620, a magnetic storage device controller module 622, and a run-time system 624.

Reagent analysis module 616 contains routines related to the specific measurement being performed. In one embodiment of the present invention, reagent analysis module 616 reads information describing the experiment from a region of memory located on the surface of the spinnable medium. In alternate embodiments of the present invention, reagent analysis module 616 accepts input from an experimenter describing the experiment and/or operation parameters for performing the experiment. Reagent analysis module 616 may also contain routines incorporating knowledge about the physical processes involved in the experiment. For example, it may calculate the magnitude of a reaction based on the amount of magnetic material measured in various parts of the disk.

Magnetic sensing driver module 618 controls an electromagnet sensing mechanism capable of measuring a spinnable medium. In one embodiment of the present invention, a sensing mechanism is derived from a read/write head of a magnetic storage device. Magnetic sensing driver module 618 initiates the measurements by sending commands to the read/write head to generate and sense magnetic fields in specified regions of the spinnable medium.

Magnetic valve actuator module 620 contains routines for controlling one or more magnetically actuated valves that present in the spinnable medium. For example, the spinnable medium may contain multiple chambers potentially connected to each other by way of one or more channels and valves. The experimenter may wish to open or close these valves at different times during an experiment or at substantially the same time interval. Magnetic valve actuator module 620 performs these operations by transmitting the appropriate instructions to the read/write head at the appropriate time periods. The read/write head in turn creates the appropriate magnetic fields in various regions on the spinnable medium to operate the nearby valves.

Magnetic storage device controller module 622 contains routines related to the motion of the spinnable medium in the rotating mechanism. For example, the experiment may require the spinnable medium to accelerate, reciprocate, or decelerate. Each of these actions affects the fluid(s) in the spinnable medium. Moreover, the read/write head of the drive must approach particular regions of the moving disk at particular moments in order to sense or affect the actions of the fluid(s).

Run-time module 624 manages system resources used when processing one or more of the previously mentioned modules. For example, the module may ensure that the magnetic valve actuator module synchronizes with the disk drive controller module and addresses the appropriate region on the spinnable medium.

System 600 can be preprogrammed, in ROM, for example, using field-programmable gate array (FPGA) technology or it can be programmed (and reprogrammed) by loading a program from another source (for example, from a floppy disk, an ordinary disk drive, a CD-ROM, or another computer). In addition, system 600 can be implemented using customized application specific integrated circuits (ASICs).

Having thus described exemplary embodiments of the present invention, it should be noted by those skilled in the art that the within disclosures are exemplary only and that various other alternatives, adaptations and modifications may be made within the scope of the present invention. For example, a variety of spinnable media and magnetic read/write mechanisms are available or will become available and could be used to embody the described invention. Various commercially available magnetic storage devices have been mentioned, but new ones continually become available. Moreover, the present can be implemented using a modified or custom-designed device.

Many arrangements of chambers and valves are possible; and many principles and valves can affect the flow of contained fluids. The term “fluid” has been used throughout, but the technique can measure reactions and characteristics of other materials, including gases, liquids, solids, or other forms of matter having magnetic properties. Some of the examples given used a single fluid. However, many embodiments are possible which process more than one fluid. The words “testing,” “experimenting,” and “characterizing” have been used throughout, but these terms are often interchangeable and no limitation on the use of the invention is implied. Moreover, “user,” “experimenter,” and other terms have been used to describe an individual utilizing or practicing the methods and systems described here, but no limitation is implied by that; and the methods and systems described here may be used for experiment or in practical applications.

Embodiments of the invention can be implemented in digital electronic circuitry, or in computer hardware, firmware, software, or in combinations of them. Apparatus of the invention can be implemented in a computer program product tangibly embodied in a machine-readable storage device for execution by a programmable processor; and method steps of the invention can be performed by a programmable processor executing a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs.

the invention is not limited to the specific embodiments described and illustrated above. Instead, the invention is construed according to the claims that follow.

What is claimed is:

1. An apparatus for characterizing reactions comprising:
a spinnable medium with one or more internal chambers capable of containing one or more reagents;
a composite reagent in one or more of the internal chambers, the composite reagent further comprising a magnetic component;
a rotating mechanism capable of turning the spinnable medium; and
a reading mechanism capable of measuring the magnetic component at one or more regions of the spinnable medium.

2. The apparatus of claim 1, wherein the spinnable medium has substantially the shape of a disk.

3. The apparatus of claim 1, wherein the internal chambers are capable of holding fluids for microfluidic testing of reagent materials.

4. The apparatus of claim 2, wherein the spinnable medium has substantially the dimensions of a commercially available removable magnetic information storage disk.

5. The apparatus of claim 4, wherein the rotating mechanism and the reading mechanism are contained in a commercially available removable magnetic information storage device.

6. The apparatus of claim 5, wherein the commercially available removable magnetic information storage device is selected from a set of devices including: a removable hard-drive, a floppy disk drive, and a magneto-optic drive.

7. The apparatus of claim 1, wherein the composite reagent further comprises functionalized magnetic beads selected from a set of functionalized magnetic beads including: functionalized ferromagnetic beads, and functionalized paramagnetic beads.

8. The apparatus of claim 7, wherein the composite reagent is capable of functioning as a chemical sandwich assay.

9. A method of characterizing reactions in a spinnable medium, comprising:
placing a composite reagent into a reaction chamber within a spinnable medium, the composite reagent further comprising a tethered component with a bond to a second magnetic component;
adding to the reaction chamber a target reagent that may react with the tethered component and displace the second magnetic component;
operating the spinnable medium to facilitate transfer of the second magnetic component to an output chamber if it is displaced;
measuring the second magnetic component; and
characterizing the reaction of the target reagent and the tethered component according to the measurement of the second magnetic component.

10. The method of claim 9, wherein the composite reagent further comprises chemical structures selected from a set of chemical structures including: antigens, expressed sequence tags, cDNAs, proteins, secondary antigen particles, nucleic acids, and amine-terminated particles.

11. The method of claim 9, wherein the spinnable medium has substantially the dimensions of a commercially available information storage device.

12. The method of claim 11, wherein measuring the second magnetic component further comprises measuring the amount of the second magnetic component in the output chamber.

13. The method of claim 11, wherein measuring the second magnetic component further comprises measuring the amount of the second magnetic component in the reaction chamber.

14. The method of claim 9, wherein the composite reagent is a sandwich assay using a chemical bond between the tethered component and the second magnetic component.

15. The method of claim 14, wherein placing the composite reagent further comprises producing the composite reagent by:
receiving the tethered component in the reaction chamber; and
introducing the second magnetic component into the reaction chamber so that it forms a chemical bond with the tethered component.

16. The method of claim 14, wherein the composite reagent is placed in the reaction chamber when the disk is manufactured.

17. The method of claim 9, wherein operating the disk includes a sequence of one or more operations selected from a set including: starting the rotation of the disk, reciprocating, fully rotating, accelerating, decelerating, and stopping rotation of the disk.

18. The method of claim 9, wherein the measuring of the second magnetic component is performed using a magnetic read head compatible with reading a commercially available removable magnetic information storage disk.

19. The method of claim 9, wherein the magnetic component is transferred to an output chamber by centrifugal force if the composite reagent reacts with the target reagent.

20. The method of claim 19, wherein the characterization determines the degree of reaction between the target reagent and composite reagent based on the amount of the second magnetic component measured in the output chamber.

21. A method of regulating the flow of fluids in a spinnable medium, comprising:
inserting a magnetic material into one or more valve areas that separate one or more channels capable of carrying fluids in a spinnable medium;
selectively introducing a magnetic field gradient in the vicinity of one or more of the valve areas to displace the magnetic material associated with one or more of the valve areas;
opening a connection between one or more of the channels responsive to the displacement of the magnetic material; and
rotating the spinnable medium thereby causing one or more fluids to flow through the valve areas under the influence of centrifugal force.

22. The method of claim 21, wherein the magnetic material is a viscous ferrofluidic material having embedded ferrous particulate selected from a set of ferrous material including: Iron (Fe), Cobalt (Co), Nickel, Ferrous-Oxide, or Fe₃O₄ or their alloys.

23. The method of claim 21, wherein the valve areas are created by directly plugging an area between the input channel and the one or more output channels with the magnetic material.

24. The method of claim 21, wherein the valve areas operate under indirect control of the ferrofluidic material moving and creating a vacuum that moves solid plugs in the valve areas.

25. The method of claim 21, wherein the valve areas operate an experimental function selected from a set of experimental functions including: flow sequencing; cascade micro-mixing; and capillary metering.

26. An apparatus for characterizing reactions comprising:
means for placing a composite reagent into a reaction chamber within a spinnable medium, the composite reagent further comprising a tethered component with a bond to a second magnetic component;
means for adding to the reaction chamber a target reagent that may react with the tethered component and displace the second magnetic component;
means for operating the spinnable medium to facilitate transfer of the second magnetic component to an output chamber if it is displaced; and
means for measuring the second magnetic component.
